(19) United States
(12) Patent Application Publication
(10) Pub. No.: US 2026/0102421 A1
(43) Pub. Date: Apr. 16, 2026

(54) Method of Treatment of SARS-CoV2 and Lewy-Body Spectrum Dementia Diseases

(71) Applicant: Glen Benjamin Van Atta, JR., Lima, OH (US)

(72) Inventor: Glen Benjamin Van Atta, JR., Lima, OH (US)

(21) Appl. No.: 19/405,338

(22) Filed: Dec. 1, 2025

Related U.S. Application Data
(60) Provisional application No. 63/732,868, filed on Oct. 1, 2024.

(51) Int. Cl. A61K 31/7048; A61K 31/352; A61K 31/375; A61K 31/455; A61K 31/4706; A61K 31/573; A61K 31/593; A61K 33/30; A61P 25/28; A61P 31/14

(52) U.S. Cl. CPC A61K 31/7048; A61K 31/352; A61K 31/375; A61K 31/455; A61K 31/4706; A61K 31/573; A61K 31/593; A61K 33/30; A61P 25/28; A61P 31/14

(57) ABSTRACT

A method of treatment of Sars-COV-2 viral infections including Covid-19 and long Covid and of virus associated cognitive impairment and Lewy-Body Spectrum Dementia disorders in a six-agent treatment regimen of hydroxychloroquine, azithromycin, zinc, quercetin, vitamin C, and vitamin D3. The six-agent treatment is administered to the subject orally for, preferably, 5 days at which time the subject is cured of Covid-19. In an alternant embodiment, the method comprises administering to a subject an eight-agent treatment regimen of hydroxychloroquine, azithromycin, zinc, quercetin, vitamin C, vitamin D3, pregnenolone, and niacinamide. The addition of pregnenolone, and niacinamide to the six-agent treatment enhances the improvement of cognitive performance.

### CROSS-REFERENCE TO RELATED APPLICATION

This application claims the benefit of U.S. Provisional Patent Application No. 63/732,868 filed on Oct. 1, 2024, which is incorporated herein in its entirety.

### FIELD OF THE DISCLOSURE

This disclosure relates generally to treatment of SARS-COV2 and, more particularly, to treatment of SARS-COV2 and SARS-COV2 with Lewy-Body Spectrum Dementia diseases using oral treatment regimens with safe and low side effect profiles.

### BACKGROUND OF THE DISCLOSURE

Oral drugs for the treatment of SARS-COV2 include nirmatrelvir and molnupiravir. Nirmatrelvir is associated with hives, skin rash, blisters, or peeling skin, painful sores or ulcers in the mouth, nose, throat, or genital area, trouble swallowing or breathing, swelling of the mouth, lips, tongue, or face, throat tightness, and hoarseness of voice. Molnupiravir is associated hives, rapid heartbeat, trouble swallowing or breathing, and swelling of the mouth, lips, or face. Molnupiravir has been shown to be associated with a slightly higher death rate. During the SARS-CoV-2 pandemic dementia has been identified as disproportionally common in adults aged over 65 who develop severe COVID-19. What is needed is a simple, low side effect, and relatively inexpensive therapeutic composition that is effective in the treatment of SARS-COV2 and in the treatment of SARS-COV2 associated with Lewy-Body Spectrum Dementia diseases.

### SUMMARY OF THE DISCLOSURE

The method of treatment of Sars-COV-2 viral infections (including Covid-19 and long Covid) includes administering to a subject in need thereof a six-agent treatment regimen of hydroxychloroquine, azithromycin, zinc, quercetin, vitamin C, and vitamin D3. The six-agent treatment is administered to the subject orally for 4-6 days, preferably 5 days.

In an alternant embodiment, a method of treatment of Sars-COV-2 viral infections including Covid-19 and long Covid and of virus associated cognitive impairment and Lewy-Body Spectrum Dementia disorders thereof comprises administering to a subject an eight-agent treatment regimen of hydroxychloroquine, azithromycin, zinc, quercetin, vitamin C, vitamin D3, pregnenolone, and niacinamide.

Hydroxychloroquine, azithromycin, zinc, quercetin, vitamin C, and vitamin D3 are administered for three months on days 1-5, 11-15, and 21-25 for each of the three months and administering hydroxychloroquine, azithromycin, zinc, quercetin, vitamin C, vitamin D3, pregnenolone, and niacinamide for said three months on days 6-10, 16-20, and 26 through the end of the month for each of the three months. Preferably, hydroxychloroquine, azithromycin, zinc, quercetin, vitamin C, vitamin D3, pregnenolone, and niacinamide administered to the subject orally.

### BRIEF DESCRIPTION OF THE FIGURES

FIG. 1 is a table showing a 5-day antiviral treatment regimen for the treatment of Sars-CoV-2 viral infections including Covid-19 and long Covid.

FIG. 2 is a table showing an alternant embodiment of a monthly antiviral treatment regimen for the treatment of Sars-COV-2 viral infections including Covid-19, Long Covid, and virus associated cognitive impairment and for Lewy-Body Spectrum Dementia disorders.

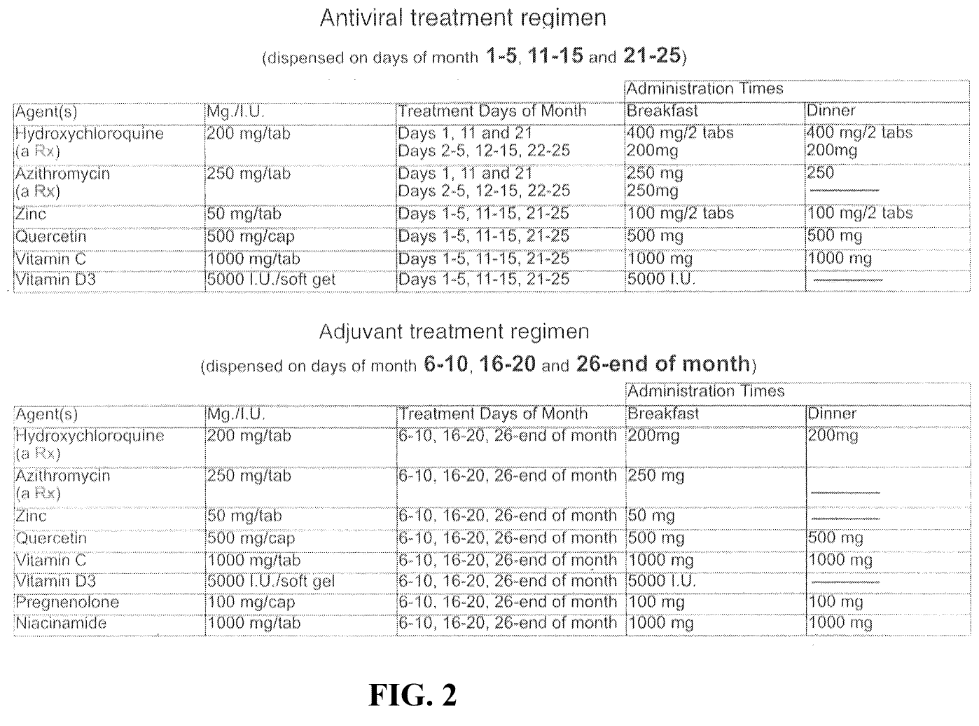

### DETAILED DESCRIPTION OF THE DISCLOSURE

While the following description details the preferred embodiments of the present disclosure, it is to be understood that the invention is not limited in its application to the details of compositions or steps of methods illustrated in the accompanying figures, since the compositions and methods are capable of other embodiments and of being practiced in various ways.

The antiviral six-agent five-day treatment regimen disclosed herein is an all oral, in home, economical, safe, and highly effective treatment for SARA-COV-2, including Covid 19, long covid, viruses associated cognitive impairment and Lewy-Body Spectrum Dementia diseases. The six-agent five-day treatment regimen has been used multiple times when the antigen self-test result was positive for Covid-19. After the treatment regimen each patient became nasal swab antigen test negative. The following is an example.

A female patient with Lewy-Body Spectrum Dementia (L.B.S.D) whose mother also had L.B.S.D was tested positive for Covid-19 by nasal swab antigen testing. The patient presented with the following progressive symptomatic history:
1) R.E.M. sleep disturbances from ages 49-59 years (was overworked and slept poorly).
2) Experienced mild obesity at age 59 years (135-170 pounds).
3) Experienced Epstein-Barr virus (E.B.V.) at age 59 years at was treated with an I.V. antiviral agent for 10 days.
4) Had fluctuations in attention and 69 years.
5) Mild paranoia at 70 years.
6) Age 71 years had disorientation, delusions, and vivid hallucinations.
7) Age 72 years lost bowel and bladder control.
8) Ages 75-77 years her verbiage became “word salad”.
9) Ages 77-79 years Parkinson's Disease symptoms were noted with flattening of affect, increasing muscle tonicity, occasional dyskinetic movements and progressive difficulty walking. Her husband retired and cared for her from ages 69-75 years. She became a profound fall risk and was placed into a dementia unit at age 75.

The patient was proven Covid-19 positive by nasal swab antigen testing at 82 years of age. She was treated with the antiviral six-agent five-day treatment regimen disclosed herein.

At the beginning of this treatment she had not spoken for seven years, had a flat affect, and increased muscle rigidity from the Parkinson's Disease. Results of treatment were as follows.
1) The patient was cured of her covid infection and she had a negative nasal swab antigen result.
2) At the end of the six-agent five-day antiviral treatment regimen the patient began to speak clearly again, which she had not done so for the previous seven years.
3) At the twelfth day after starting the five-day treatment regimen her muscle rigidity abated and her flat affect improved.
4) Her symptoms recurred slowly thereafter but she remained covid-free.

It is surmised that the patient's rather abrupt response to the five-day antiviral treatment regimen with improved cognitive functions is related to attenuation of neuroinflammation by modulating (or normalizing) the inflammatory milieu through the inhibition of proinflammatory cytokines particularly in and around the hippocampus. In view of the above findings of cognitive improvement, an eight-agent treatment was developed in which two adjuvant agents were added to the six-agent treatment to further enhance cognitive improvement. By alternating the six-agent five-day treatment antiviral regimen with an eight-agent five-day treatment antiviral cognitive enhancement regimen, repeated on a monthly basis, progressive potentiation of cognitive improvement occurred and the patient's symptoms slowly improved.

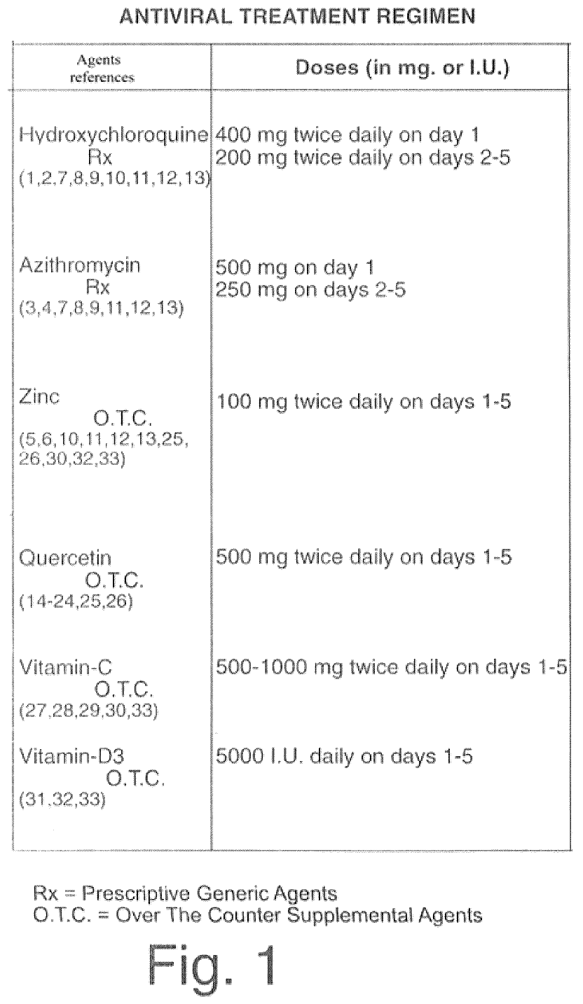

The agents used in the six-agent treatment are hydroxychloroquine, azithromycin, zinc, quercetin, vitamin C and vitamin D3. FIG. 1 lists these agents, references that describe these agents, and oral dosing conditions. Hydroxychloroquine is chaotropic agent and allows the azithromycin-zinc combination entry into cellular interiors. Hydroxychloroquine is a zinc ionophore. Azithromycin is an immunomodulatory agent and a macrolide antibiotic.

Azithromycin-zinc inhibits intracellular viral replication. Zinc is a crucial cation in numerous biochemical pathways and is a crucial structural element. Quercetin is a potent bioflavinoid anti-inflammatory, a zinc ionophore, and increases zinc absorption. Vitamin C is a zinc ionophore and decreases the risk for viral infection susceptibility. Vitamin D3 is a zinc ionophore and decreases the risk for viral infection susceptibility.

A method of treatment of Sars-COV-2 viral infections including Covid-19 and long Covid in a subject in need thereof comprises administering to the subject the six-agent therapeutic regimen of hydroxychloroquine, azithromycin, zinc, quercetin, vitamin C, and vitamin D3. The treatment is administered for four to six consecutive days, preferably for five consecutive days. Preferably, the six agents are administered to the subject by the oral route of administration (orally). Hydroxychloroquine is administered at a dosage of 300-1000 mg per day. Azithromycin is administered at a dosage of 200-600 mg per day. Zinc is administered at a dosage of 150-250 mg per day. Quercetin is administered at a dosage of 800 to 1,200 mg per day. Vitamin C is administered at a dosage 800 to 1,200 mg per day. Vitamin D3 is administered at a dosage of 4000-6000 IU per day. A subject in need thereof would be, for example, one who tested positive for Covid-19.

The method of treatment reduces pro-inflammatory activity associated with Sars-COV-2 viral infections including Covid-19 and long Covid and makes the subject nasal swab antigen test negative.

In an alternant embodiment two adjuvants, niacinamide and pregnenolone are added to the six-agent treatment to provide an eight-agent cognitive enhancement treatment of Sars-COV-2 viral infections including Covid-19 and long Covid and of virus associated cognitive impairment and Lewy-Body Spectrum Dementia disorders in a subject in need thereof. An eight-agent method of treatment comprises hydroxychloroquine, azithromycin, zinc, quercetin, vitamin C, vitamin D3, pregnenolone, and niacinamide.

Preferably, hydroxychloroquine, azithromycin, zinc, quercetin, vitamin C, and vitamin D3 are administered for three months on days 1-5, 11-15, and 21-25 for each of the three months and administering hydroxychloroquine, azithromycin, zinc, quercetin, vitamin C, vitamin D3, pregnenolone, and niacinamide for said three months on days 6-10, 16-20, and 26 through the end of the month for each of the three months. Preferably, the eight agents are administered to the subject orally.

Preferably, on days 1-5, 11-15, and 21-25 hydroxychloroquine is administered to the subject at a dosage of 300-1000 mg per day, azithromycin is administered at a dosage of 200-600 mg per day, zinc is administered at a dosage of 150-250 mg per day, quercetin is administered at a dosage of 800-1200 mg per day, vitamin C is administered at a dosage of 1,800-2,200 mg per day, and vitamin D3 is administered at a dosage of 4,000-6,000 IU per day.

Preferably, on days 6-10, 16-20, and 26-end of month, hydroxychloroquine is administered at a dosage of 305-405 mg per day, azithromycin is administered at dosage of 200-300 mg per day, zinc is administered at a dosage of 40-60 mg/day, quercetin is administered at a dosage of 800 to 1,200 mg per day, vitamin C is administered at a dosage of 1,800-2,200 IU per day, vitamin D3 is administered at a dosage of 4,000-6,000 IU per day, pregnenolone is administered at a dosage of 150-250 mg per day, and niacinamide is administered at a dosage of 1,800-2,200, mg per day.

The foregoing description illustrates and describes the antiviral treatments of the disclosure. Additionally, the disclosure shows and describes only the preferred embodiments, but it is to be understood that the preferred embodiments are capable of being formed in various other combinations, modifications, and environments and are capable of changes or modifications within the scope of the disclosure, commensurate with the above teachings and/or the skill or knowledge of the relevant art. The embodiments described herein are further intended to explain the best modes known by applicant and to enable others skilled in the art to utilize the antiviral treatments in such, or other, embodiments and with the various modifications required by the particular applications or uses thereof. Accordingly, the description is not intended to limit the disclosure to the form disclosed herein. Also, it is intended that the appended claims be construed to include alternative embodiments. It will be further understood that various changes in the details, materials, and arrangements of the parts which have been described and illustrated above in order to explain the nature of the antiviral treatments may be made by those skilled in the art without departing from the principle and scope of the disclosure as recited in the following claims.

TABLE 1

| | | | |
|---|---|---|---|
| No. | Citation | Notes | Conclusion |

HQC:

| | | | |
|---|---|---|---|
| 1 | X. Yao, et al. Clin. Infect. | “In vivo anti-viral activity and | Hydroxychloroquine |
| | Dis. 2020 Jul. 28; 71(15): 732- | projection of optimzed dosing | 400 mg bid on day 1 |
| | 739 (Epub ahead of print) | design of Hydroxychloroquine for | then 200 mg bid on |
| | | the treatment of Severe Acute | days 2-5 |
| | | Respiratory Syndrome Corona | |
| | | Virus-2 (SARS-CoV-2)” | |
| 2 | Z. Yuan, et al. | “Hydroxychloroquine blocks | — |
| | | SARS-CoV-2 entry into the | |
| | | endocytic pathway in mammallian | |
| | | cell culture.” | |

AZM:

| | | | |
|---|---|---|---|
| 3 | M. E. Oliver, et al. Rev. Med. | “Azithromycin in viral infections.” | — |
| | Virol. 2021; 31: e2163 | | |
| 4 | J. A. Kricker, et al. Pharmacol. | “Nonantimicrobial actions of | — |
| | Rev. 2021 October; 73: 1404-1433 | macrolides: overview and | |
| | | perspectives for future | |
| | | development.” | |

ZN++:

| | | | |
|---|---|---|---|
| 5 | A. J. te Velthuis, et al. | “Zinc (ZN++) inhibits Coronavirus | — |
| | Pathogens 2010; 6(11) | and Arterivirus RNA polymerase | |
| | | activity in vitro and Zinc ionophores | |
| | | block the replication of these viruses | |
| | | in cell culture.” | |
| 6 | S. A. Read, et al. Advances in | “The role of Zinc in anti-viral | — |
| | Nutrition 2019; 10(4): 696-710 | immunity.” | |

HQC/AZM:

| | | | |
|---|---|---|---|
| 7 | H. A. Risch. Amer. J. | “Early out-patient treatment of | Hydroxychloroquine + |
| | Epidemiol. 2020 Nov. | symptomatic high risk Covid-19 | Azithromycin |
| | 2; 189(11): 1218-1226 (Epub | patients that should be ramped-up | outpatient treatment; |
| | May 27, 2020 ahead of print) | immediately as key to the pandemic | prior studies show |
| | | crisis.” | “significant major |
| | | | outpatient treatment |
| | | | efficacy.” |
| 8 | M. Million, et al. Travel Med. | “Early treatment of Covid-19 | — |
| | Infect. Dis. 2020 May- | patients with Hydroxychloroquine | |
| | June; 35: 101738 (Epub May 5, | and Azithromycin: a retrospective | |
| | 2020) | analysis of 1061 cases in Marseilles, | |
| | | France.” | |
| 9 | E. R. Barbosa, et al. (via | “Empirical treatment with | — |
| | Pgibertie.files.wordpress.com, | Hydroxychloroquine and | |
| | Epub Apr. 30, 2020) | Azithromycin for suspected cases of | |
| | | Covid-19 follow-up by telemedicine | |
| | | (in São Paulo, Brazil in 636 | |
| | | symptomatic high-risk outpatients | |
| | | monitored via telemedicine daily).” | |

HQC/ZN++:

| | | | |
|---|---|---|---|
| 10 | R. Derwand, et al. Medical | “Zinc supplementation enhances | — |
| | Hypothesis 2020; 142: 109815 | the clinical efficacy of | |
| | (Epub ahead of print) | hydroxychloroquine to win the | |
| | | Covid-19 battle.” | |

AZM/ZN++

| | | | |
|---|---|---|---|
| 11 | J. H. Poupaert, et al. The Open | “Study of the interaction of Zinc | — |
| | Biochemistry J. 2020 Sep. 22 | cation with Azithromycin and its | |
| | (Epub ahead of print) | significance in the Covid-19 | |
| | | treatment: A molecular approach.” | |

HQC/AZM/ZN++:

| | | | |
|---|---|---|---|
| 12 | Y. Zelenko. Letter (2-page | “To all medical professionals | “excellent efficacy in |
| | report), Apr. 28, 2020. Epub | around the world.” | outpatient high-risk |
| | via docs.google.com | | patients.” |
| 13 | R. Derwand, et al. Int. J. | “Covid-19 outpatients: early risk- | — |
| | Antimicrob. Agents 2020 | stratified treatment with Zinc plus | |
| | December; 56(6): 10624 (P.O.L.: | low dose Hydroxychloroquine and | |
| | Oct. 26, 2020) | Azithromycin: a retrospective use | |
| | | series study.” | |

QCT:

| | | | |
|---|---|---|---|
| 14 | C. J. Chen, et al. | “Toona sinesis Roem tender leaf | It contains the |
| | Ethnopharmacol | extract inhibits SARS Coronavirus | flavonoid quercetin |
| | 2008; 120: 108-111 | replication.” | |
| 15 | B. Kinker, et al. J. Ancient | “Quercetin: A promising treatment | — |
| | Dis. Prev. Rem. 2014; 02(02) | for the common cold.” | |
| 16 | W. Wu, et al. Viruses | “Quercetin as an anti-viral agent | — |
| | 2015; 8(1): 6 | inhibits Influenza A Virus (IAV) | |
| | | entry.” | |
| 17 | S. Jo, et al. Chem. Biol. Drug | “Characteristics of flavonoids as | — |
| | Des. 2019 December; 94(6): | potent MERS-CoV 3c-like protease | |
| | 2023-2030 | inhibitors.” | |
| 18 | S. Jo, et al. J. Enzyme Inhib. | “Inhibition of SARS-CoV 3CL | — |
| | Med. Chem. 2020; 35: 145-151 | protease by flavonoids.” | |
| 19 | Y. Yang, et al. Int. J. Biol. | “Traditional Chinese medicine in | — |
| | Sci. 2020; 16(10): 1708-1717 | the treatment of patients infected | |
| | | with 2019-new Corona Virus | |
| | | (SARS-CoV-2): a review and | |
| | | perspective.” | |
| 20 | O. Hasan, et al. | “Effect of quercetin on prophylaxis | — |
| | clinicaltrials.gov Identifier | and treatment of CoVid-19.” | |
| | NCT04377789 (Trial: Mar. | | |
| | 20, 2020-Jul. 31, 2020) | | |
| 21 | P. K. Agrawal, et al. Natrl. | “Quercetin anti-viral significance | QCT + VIT-C + VIT-D3 |
| | Prod. Commun. 2020; 15(12): | and possible CoVid-19 integrative | |
| | 1-10 (P.O.L. Dec. 3, 2020) | considerations.” (=OCT + Vit-C + | |
| | | Vit-D3) | |
| 22 | F. D. Pierro, et al. Int. J. Gen. | “Possible therapeutic effects of | — |
| | Med. 2021; 14: 2359-2366 | adjuvant quercetin supplementation | |
| | | against early stage CoVid-19 | |
| | | infection: A prospective | |
| | | randomized, controlled, and open | |
| | | label study.” | |
| 23 | A. D. Petrillo, et al. Phytother. | “Quercetin and its derivatives as | — |
| | Res. 2022 January; 36(1): 266-278 | antiviral potentials: A | |
| | (P.O.L. Oct. 28, 2021) | comprehensive review.” | |
| 24 | A. Gasmi, et al. | “Quercetin in the prevention and | — |
| | Pharmaceuticals 2022; 15: 1049 | treatment of coronavirus infections: | |
| | | A focus on SARS-CoV-2.” | |

QCT/ZN:

| | | | |
|---|---|---|---|
| 25 | H. Dabbagh-Bazarbachi, et al. | “Zinc ionophoric activity of | — |
| | Agric. Food Chem 2014 Aug. | quercetin.” | |
| | 13; 62(32): 8085-8093 | | |
| 26 | C. Clergeaud. Food Chem. | “A simple liposome assay for the | — |
| | 2016; 197(Pt A): 916-923 | screening of zinc ionophore activity | |
| | | of polyphenols.” | |

VIT-C:

| | | | |
|---|---|---|---|
| 27 | T. Teafatiller, et al. | “Vitamin C enhances anti-viral | — |
| | Biomolecules 2021; 11: 1148 | functions of lung epithelial cells.” | |
| 28 | J. R. Miranda-Massari, et al. | “The effects of vitamin C on the | — |
| | Life 2021; 11: 1341 | multiple pathophysiological stasis of | |
| | | CoVid-19.” | |
| 29 | M. Olczak-Pruc, et al. | “Vitamin C supplementation for the | in therapeutic doses |
| | Nutrients 2022 Oct. 14; | treatment of Covid-19: A systematic | |
| | 14(19): 4217 (P.O.L. Oct. | review and meta-analysis.” | |
| | 10, 2022) | | |

VIT-C/ZN++:

| | | | |
|---|---|---|---|
| 30 | E. S. Wintergerst. Ann. Nutr. | “Immune-enhancing role of | — |
| | Chem. 2006; 50(2): 85-94 | Vitamin C and Zinc and effect on | |
| | | clinical conditions.” | |

VIT-D

| | | | |
|---|---|---|---|
| 31 | K. Shah, et al. Q. J. M. (Intnl. | “Does Vitamin D supplementation | — |
| | J. Med.) 2022 Oct. 25; 115(10): | reduce CoVid-19 severity?: A | |
| | 665-672 | systematic review.” | |

VIT-D/ZN++:

| | | | |
|---|---|---|---|
| 32 | A. Amos, et al. Curr. Res. | “Zinc and its role in Vitamin D | — |
| | Physiol. 2022; 7: 203-207 | function.” | |

VIT-C/VIT-D/ZN++:

| | | | |
|---|---|---|---|
| 33 | J. Joao, et al. Front. Nutr. | “Zinc, Vit. D and Vit. C: | — |
| | (Nutr. Immunol.) 2020 Dec. 6; 7 | Perspective for CoVid-19 with a | |
| | | focus on physical tissue barrier | |
| | | technology.” | |

What is claimed is:

1. A method of treatment of Sars-COV-2 viral infections including Covid-19 and long Covid and of virus associated cognitive impairment and Lewy-Body Spectrum Dementia disorders in a subject in need thereof, the method comprising administering to the subject hydroxychloroquine, azithromycin, zinc, quercetin, vitamin C, and vitamin D3 daily for 4 to 6 days.

2. The method of claim 1 wherein the treatment is administered to the subject orally.

3. The method of claim 2 wherein hydroxychloroquine is administered at a dosage of 200-400 mg per day, azithromycin is administered at a dosage of 250-500 mg per day, zinc is administered at a dosage of 50-100 mg per day, quercetin is administered at a dosage of 500 mg twice daily, vitamin C is administered at a dosage of 1000 mg twice daily and vitamin D3 is administered at a dosage of 5000 IU daily.

4. The method of claim 3, further comprising reducing pro-inflammatory activity associated with Sars-COV-2 viral infections including Covid-19 and long Covid.

5. The method of claim 4 further comprising making the subject nasal swab antigen test negative.

6. A method of treatment of Sars-COV-2 viral infections including Covid-19 and long Covid and of virus associated cognitive impairment and Lewy-Body Spectrum Dementia disorders in a subject in need thereof, the method comprising administering to the subject hydroxychloroquine, azithromycin, zinc, quercetin, vitamin C, vitamin D3, pregnenolone, and niacinamide.

7. The method of claim 6 wherein hydroxychloroquine, azithromycin, zinc, quercetin, vitamin C, and vitamin D3 are administered for three months on days 1-5, 11-15, and 21-25 for each of the three months and administering hydroxychloroquine, azithromycin, zinc, quercetin, vitamin C, vitamin D3, pregnenolone, and niacinamide for said three months on days 6-10, 16-20, and 26 through the end of the month for each of the three months.

8. The method of claim 7 wherein hydroxychloroquine, azithromycin, zinc, quercetin, vitamin C, vitamin D3, pregnenolone, and niacinamide administered to the subject orally.

9. The method of claim 8 wherein on days 1-5, 11-15, and 21-25 hydroxychloroquine is administered to the subject at a dosage of 300-1000 mg per day, azithromycin is administered at a dosage of 200-600 mg per day, zinc is administered at a dosage of 150-250 mg per day, quercetin is administered at a dosage of 800-1200 mg per day, vitamin C is administered at a dosage of 1,800-2,200 mg per day, and vitamin D3 is administered at a dosage of 4,000-6,000 IU per day and wherein on days 6-10, 16-20, and 26-end of month, hydroxychloroquine is administered at a dosage of 305-405 mg per day, azithromycin is administered at dosage of 200-300 mg per day, zinc is administered at a dosage of 40-60 mg/day, quercetin is administered at a dosage of 800 to 1,200 mg per day, vitamin C is administered at a dosage of 1,800-2,200 IU per day, vitamin D3 is administered at a dosage of 4,000-6,000 IU per day, pregnenolone is administered at a dosage of 150-250 mg per day, and niacinamide is administered at a dosage of 1,800-2,200, mg per day.

10. The method of claim 9 further comprising reducing in the subject pro-inflammatory cytokine activity associated with SARS-COV-2 infections and Lewy-Body Spectrum Dementia disorders.

11. The method of claim 10 further comprising improving in the subject cognitive performance and reducing symptoms of brain fog.

12. The method of claim 11 further comprising making the subject nasal swab antigen test negative.
